(19) United States
(12) Patent Application Publication
(10) Pub. No.: US 2016/0120375 A1
(43) Pub. Date: May 5, 2016

(54) Dual-Function Skin Cleansing Apparatus

(71) Applicant: Epicare, Ltd., New York, NY (US)

(72) Inventor: Evan J. Philips, New York, NY (US)

(21) Appl. No.: 14/531,654

(22) Filed: Nov. 3, 2014

(51) Int. Cl. A47K 7/04; A61B 17/54; A61H 23/00; A61H 15/00

(52) U.S. Cl. CPC A47K 7/043; A61H 15/0092; A61B 17/54; A61H 23/00; A61B 2017/00526

(57) ABSTRACT

Embodiments of the present invention relate a dual function cleansing apparatus. Specifically, the present invention relates to embodiments of a facial cleansing apparatus having one or more brushes that may have a rapid rotation or an oscillation. More specifically, embodiments of the present invention include a facial cleansing device having a rotating brush and an oscillating brush. In a preferred embodiment, the rotating brush has a rotating motion, and the oscillating brush has an oscillating motion that is driven by a single motor.

### TECHNICAL FIELD

The present invention relates to embodiments of a dual function skin cleansing apparatus. Specifically, the present invention relates to embodiments of a skin cleansing apparatus having one or more brushes that may have a rapid rotation or an oscillation. More specifically, embodiments of the present invention include a skin cleansing device having a rotating brush and an oscillating brush. In a preferred embodiment, the rotating brush has a rotating motion, and the oscillating brush has an oscillating motion that is driven by a single motor.

### BACKGROUND

Skin cleansing, and especially facial cleansing, is an important aspect of proper hygiene. Indeed, dirt, pollution, tobacco, make-up and excess oils may lead to unhealthy skin unless properly washed. Fatigue and stress may also contribute to unhealthy facial skin. These factors can promote bacterial growth, further leading to blemishes, acne, wrinkles, dry spots, flaky skin, and other similar skin maladies.

Facial cleansers are well-known, and may take the form of one or a combination of salves, creams, ointments, soaps, medicines and other like compositions that remove the impurities and/or kill undesired bacteria on or in facial skin. A medicinal ointment, for example, may include an amount of salicylic acid, which may be used to fight bacterial growth and reduce blemishes and acne. Other similar compositions may be used to cleanse the surface of the skin, as well as the pores of the skin. In addition, moisturizers may be used to hydrate the skin of the face, further promoting healthy facial skin.

Apparatuses are also known to aid in cleansing skin, which may range from fairly simple items to more complex. For example, a washcloth, which is simply a piece of soft, yet mildly abrasive fabric, may be used in combination with soaps or other cleansing composition and may aid in scrubbing skin to remove dirt and oils from the skin and from the pores thereof. However, simple washcloths fail to provide an effective facial skin cleansing solution, and oftentimes leave behind dirt and oils, which may contribute to unhealthy skin. Moreover, since a user must provide the scrubbing action of a washcloth, oftentimes the users scrub too hard, which can irritate or even damage relatively sensitive skin, or too soft, reducing the effectiveness of the washcloth. A need, therefore, exists for a skin cleansing apparatus that effectively aids in the removal of dirt, oils, make-up, and other like pollutants that may promote unhealthy skin. Moreover, a need exists for a skin cleansing apparatus that is gentle on relatively sensitive skin, preventing skin irritation and damage thereto.

More complex skin cleansers include devices that may provide brushes or pads that aid in cleansing skin, especially when used in conjunction with soaps and other cleansing compositions. A common skin cleansing apparatus includes a device having a rotating pad or brush that may be driven by a motor. Such devices may be powered using standard AC power (such as wall power) or DC power (such as battery power). AC powered skin cleansing devices must further be plugged directly into an AC power source, and as such are limited in their usage and range. DC powered skin cleansing devices must include space for battery packs and as such can be bulky and difficult to handle. A need, therefore, exists for an exclusively DC powered skin cleansing device that is powered from a DC battery pack. However, a need further exists for a DC powered skin cleansing device that is sleek, lightweight, and easy to handle and manipulate.

Vibrating devices are also known for use with skin cleansing. Typically, a vibrating device utilizes an off-center weight that may spin about a rod extending from a motor. The rapid spinning of the off-center weight causes the entirety of the device to vibrate, which may cause a cleansing pad or brush to likewise vibrate. While the vibratory motion may promote skin cleansing, the vibrating is often difficult to control, and is generally ineffective in providing enhanced cleansing of a user's skin.

Other skin cleansing apparatuses utilize a pad or brush having an oscillating motion that may provide the benefits of vibratory motion, but may be gentler on sensitive skin, and easier to control. The oscillating motion further may be directed specifically to the brush or pad, instead of to the entirety of the device, such as with the vibratory motion described above.

Heretofore, skin cleansing devices have a pad or a brush that has either rotating motion or oscillating motion, but there is presently no single device that provides, alternately, rotating motion and oscillating motion. No prior skin cleansing apparatus combines both of the motions of rapid rotation and oscillation in a single device to allow a user to obtain the benefits of both motions. Some skin cleansing devices combine rotating motion with vibratory motion, as described above, but these devices are awkwardly designed, difficult to handle, hard to control, and are generally ineffective at cleansing skin. However, as noted, vibratory motion of the device is not the same as oscillating motion of the pad or brush. A need, therefore, exists for a skin cleansing apparatus combining the dual motions of rapid rotation of a pad or brush, and oscillating motion of the pad or brush. More specifically, a need exists for a skin cleansing apparatus providing both rapid rotation and oscillation of one or more pads or brushes from a single motor, in an easy-to-handle apparatus that is simple to control.

### SUMMARY OF THE INVENTION

The present invention relates to embodiments of a dual function skin cleansing apparatus. Specifically, the present invention relates to embodiments of a skin cleansing apparatus having one or more brushes that may have a rapid rotation or an oscillation. More specifically, embodiments of the present invention include a skin cleansing device having a rotating brush and an oscillating brush. In a preferred embodiment, the rotating brush has a rotating motion, and the oscillating brush has an oscillating motion that is driven by a single motor.

To this end, in an embodiment of the present invention, a skin cleansing apparatus is provided. The skin cleansing apparatus comprises: a main body having a power source and a cleansing head on an end thereof, the cleansing head comprising an oscillating plate and a rotating axle for separately driving a cleansing attachment in either an oscillation motion or a rotating motion.

In an embodiment, the skin cleansing apparatus further comprises: a battery enclosure within the main body for providing DC power.

In an embodiment, the skin cleansing apparatus further comprising: a motor, the motor interconnected to both the oscillating plate and the rotating axle for driving both the oscillating plate and the rotating axle.

In an embodiment, the skin cleansing apparatus further comprises: a power switch for engaging the motor.

In an embodiment, the skin cleansing apparatus further comprises: a set of mechanical elements to translate rotational motion of the motor into oscillating motion of the oscillating plate.

In an embodiment, the skin cleansing apparatus further comprises: a set of mechanical elements to drive rotational motion of the rotating axle.

In an embodiment, the skin cleansing apparatus further comprises: a cleansing attachment engaging the oscillating plate.

In an embodiment, the cleansing attachment engaging the oscillating plate has an abrasive surface for cleansing a user's skin.

In an embodiment, the skin cleansing apparatus further comprises: a cleansing attachment engaging the rotating axle.

In an embodiment, the cleansing attachment engaging the rotating axle has an abrasive surface for cleansing a user's skin.

In an embodiment, the motor drives the oscillation at a first oscillating speed and the rotation at a first rotating speed.

In an embodiment, the motor drives the oscillation at a second oscillating speed and the rotation at a second rotating speed.

In an embodiment, the motor drives the oscillation between about 14,000 and 20,000 oscillations per minute and the rotation between about 250 and 400 rotations per minute.

In an embodiment, the first oscillation speed is about 15,000 oscillations per minute and the first rotation speed is about 360 rpm.

In an embodiment, the first oscillating speed is about 14,000 oscillations per minute and the second oscillating speed is between about 17,000 and 20,000 oscillations per minute, and the first rotating speed is between about 280 and 320 rpm and the second rotating speed is between about 355 and 395 rpm.

In an alternate embodiment of the present invention, a method of cleansing skin is provided. The method comprises the steps of: providing a skin cleansing apparatus comprising a main body having a power source and a cleansing head on an end thereof, the cleansing head comprising an oscillating plate and a rotating axle for separately driving a cleansing attachment in either an oscillation motion or a rotating motion; and attaching a first cleansing attachment to the oscillating plate for oscillating the first cleansing attachment.

In an embodiment, the method further comprises the step of: attaching a second cleansing attachment to the rotating axle for rotating the second cleansing attachment.

In an embodiment, the cleansing attachment comprises a cleansing surface.

In another alternate embodiment of the present invention, a method of cleansing skin is provided. The method comprises the steps of: providing a skin cleansing apparatus comprising a main body having a power source and a cleansing head on an end thereof, the cleansing head comprising an oscillating plate and a rotating axle for separately driving a cleansing attachment in either an oscillation motion or a rotating motion; and attaching a first cleansing attachment to the rotating axle for rotating the first cleansing attachment.

In an embodiment, the method further comprises the step of: attaching a second cleansing attachment to the oscillating plate for oscillating the second cleansing attachment.

It is, therefore, an advantage and objective of the present invention to provide a skin cleansing apparatus that effectively aids in the removal of dirt, oils, make-up, and other like pollutants that may promote unhealthy skin.

Moreover, it is an advantage and objective of the present invention to provide a skin cleansing apparatus that is gentle on relatively sensitive facial skin, preventing skin irritation and damage thereto.

Further, it is an advantage and objective of the present invention to provide an exclusively DC powered skin cleansing device that is powered from a DC battery pack.

Still further, it is an advantage and objective of the present invention to provide a DC powered skin cleansing device that is sleek, lightweight, and easy to handle and manipulate.

In addition, it is an advantage and objective of the present invention to provide a skin cleansing apparatus combining the dual motions of rapid rotation of a pad or brush, and oscillating motion of the pad or brush.

More specifically, it is an advantage and objective of the present invention to provide a skin cleansing apparatus providing both rapid rotation and oscillation of one or more pads or brushes from a single motor, in an easy-to-handle apparatus that is simple to control.

Additional features and advantages of the present invention are described in, and will be apparent from, the detailed description of the presently preferred embodiments and from the drawings.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 illustrates a front view of a dual-function skin cleansing apparatus having a skin cleansing brush in an embodiment of the present invention.

FIG. 2 illustrates a front view of a dual-function skin cleansing apparatus without a skin cleansing brush in an embodiment of the present invention.

FIG. 3 illustrates a perspective view of a dual-function cleansing apparatus having a skin cleansing brush attached thereto in an embodiment of the present invention.

FIG. 4 illustrates an exploded view of a dual-function cleansing apparatus with a skin cleansing brush in an embodiment of the present invention.

### DETAILED DESCRIPTION OF THE PRESENTLY PREFERRED EMBODIMENTS

The present invention relates to embodiments of a dual function cleansing apparatus. Specifically, the present invention relates to embodiments of a skin cleansing apparatus having one or more brushes that may have a rapid rotation or an oscillation. More specifically, embodiments of the present invention include a skin cleansing device having a rotating brush and an oscillating brush. In a preferred embodiment, the rotating brush has a rotating motion, and the oscillating brush has an oscillating motion that is driven by a single motor.

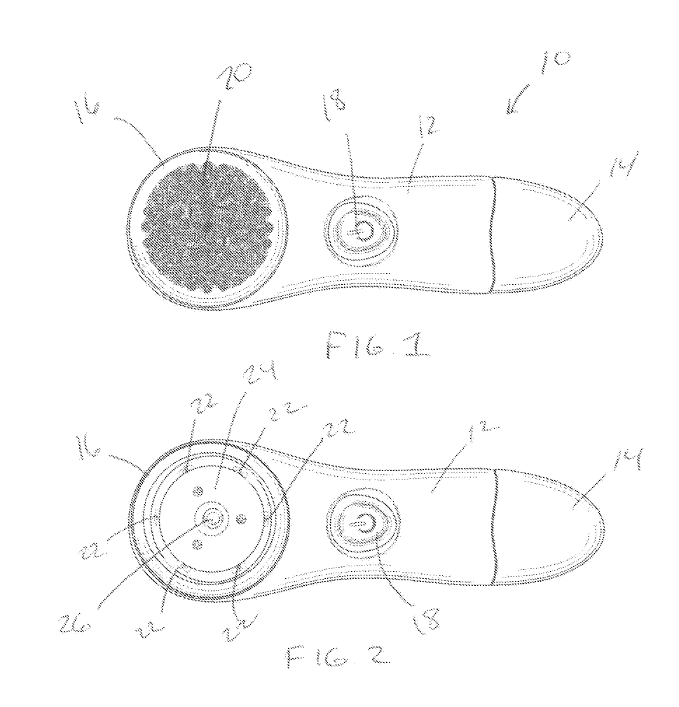

Now referring to the figures, wherein like numerals refer to like parts, FIG. 1 illustrates a front view of a dual-function skin cleansing apparatus 10 in an embodiment of the present invention that may be used on a user's body and/or face. The skin cleansing apparatus 10 comprises a main body 12 having a removable battery enclosure 14 on a first end of the main body 12, and a cleansing head 16 on a second end of the main body 12. The removable battery enclosure 14 may house one or more batteries for providing power to the apparatus 10, as needed for the functions described herein. A power button 18 may be provided on the main body 12 for powering the apparatus 10, and/or for changing the functions thereof, such as the speed of rotation and/or the speed of oscillation of an attached brush, pad or other cleansing surface, as described in more detail below. Of course, it should be noted that any number of buttons, slides, switches, lights, controllers or other control mechanisms may be provided on the main body 12, or on any other location of the apparatus 10, for controlling the various functions of the apparatus 10. In a preferred embodiment, the power button 18 simply powers the apparatus 10 without providing the ability to change speeds or rotation or oscillation. However, in other embodiments, the power button 18 may change the speeds of rotation and oscillation based on the number of times the power button 18 is pushed.

A removable skin cleansing brush 20 may be disposed on the cleansing head 16 for contacting a user's skin, such as the user's face. It should be noted that the cleansing brush 20 may take the form of a relatively smooth or relatively abrasive pad, sponge, stone, massage surface or other like surface, instead of having bristles thereon, and the invention should not be limited as described herein. Thus, the use of the term “cleansing brush” is meant to include any pad, sponge, stone, massage surface or other like surface that may be attached to the apparatus 10, as described herein.

The cleansing brush 20 may generally comprise a plurality of bristles extending from a disk-shaped holder that may be removably attachable to the cleansing head 16. The plurality of bristles may be relatively soft so as to engage a user's skin without causing irritation or damage thereto. In addition, the plurality of bristles may include combinations of relatively soft and relatively stiff bristles for increasing or decreasing abrasiveness with a user's skin, as may be apparent to one of ordinary skill in the art.

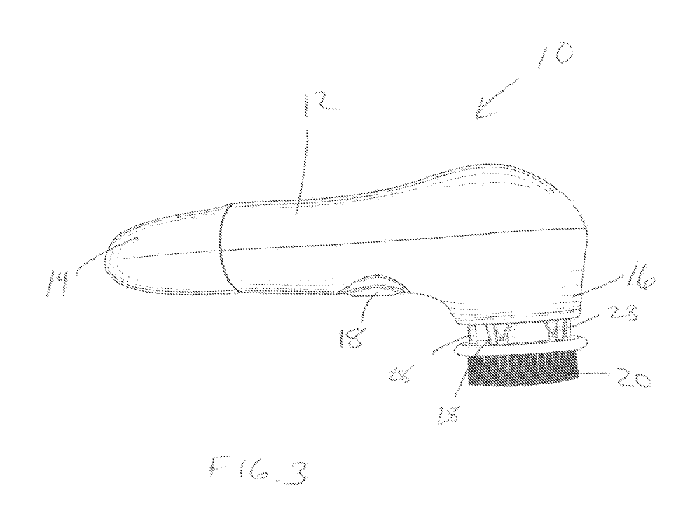

As illustrated in FIG. 2, the main body 12 is illustrated without the cleansing brush 20, thereby showing the cleansing head 16 in greater detail. The cleansing head 16 may have an oscillating plate 24 comprising a plurality of engaging elements 22 that may engage mating elements contained on a back side of the cleansing brush 20. Further, the cleansing head 16 may have a rotating axle 26 disposed centrally for engaging a similarly positioned engaging mechanism on the back side of the cleansing brush 20. The cleansing brush 20 may engage either the oscillating plate 24 or the rotating axle 26, depending on whether the oscillating function is desired or the rotating function is desired.

Alternatively, there may be a plurality of cleansing brushes 20, each of which may separately engage either the oscillating plate 24 or the rotating axle 26. For example, a first brush (not shown) may be utilized having a means for engaging only the rotating axle 26. The first brush thus may be used for only the rotating function of the apparatus 10. A second brush (not shown) may be utilized having a means for engaging only the oscillating plate 24. The second brush may be used for only the oscillating function of the apparatus 10. Many different brushes, pads, sponges, or other like surfaces may be alternately attached to the oscillating plate 24 or the rotating axle 26, depending on the desired use of the particular surface.

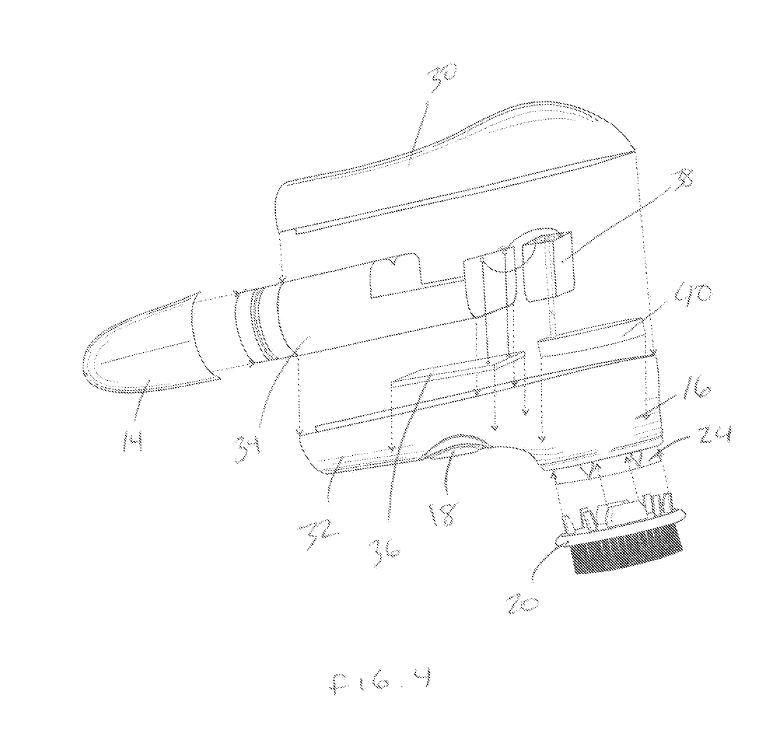

FIG. 3 illustrates a perspective view of a skin cleansing apparatus 10 in an embodiment of the present invention. The apparatus 10 comprises the main body 12, the removable battery enclosure 14 on a first side thereof, the cleansing head 16 disposed on a second side thereof, and the power button 18 disposed thereon for controlling the various functions of the cleansing apparatus 10. The removable brush 20 is further shown removably attached to the cleansing head 16 and extending therefrom, connected to the cleansing head 16 via a plurality of engaging legs 28 that engage with engaging elements 22 of the oscillating plate 24, as shown in FIG. 2. Alternatively, the cleansing brush 20 may engage the rotating axle 26, thereby providing the cleansing brush with rotating functionality. In use, the cleansing brush 20 engages a user's skin, and may be used for cleansing the user's skin when utilized with soap, salves, medicines, creams, lotions, water or other like cleansing compositions.

FIG. 4 illustrates an exploded view of the apparatus 10 in an embodiment of the present invention, showing, generally, various aspects of the internal workings of the apparatus 10. Specifically, the apparatus 10 may include a first half shell portion 30 and a second half shell portion 32. The first half shell portion 30 may form a majority of a rear side of the apparatus 10 and the second half shell portion 32 may form a majority of the front side of the apparatus 10, further containing both the power button 18 and the cleansing head 16.

The battery enclosure 14 may extend from a battery pack 34 that may be contained within the apparatus 10, and may further form a handle that may be used by a user to hold the apparatus 10 when in use. The first half shell portion 30, the second half shell portion 32, and the battery enclosure 14 may form the apparatus, forming a watertight seal to prevent water from entering the apparatus 10 and damaging internal components thereof. The battery pack 34 may contain one or more batteries for powering the apparatus 10.

The power button 18 may engage a circuit board 36 that is generally shown positionally adjacent to the power button 18 for engagement with the power button 18. The circuit board 36 may control the functions of the apparatus 10. In a preferred embodiment, the power button 18 simply powers the apparatus 10, engaging both the oscillating plate 24 and the rotating axle 26 so that either function can be utilized depending on the type of brush that is mated to the cleansing head 16 of the apparatus 10.

When engaged by the power button 18, the circuit board may control, via power supplied by the battery pack 34, a motor 38 that may be mounted within the apparatus 10. The motor 38 may engage a gear box 40 having a plurality of gears, rods and other like mechanisms for controlling both oscillation of the base 24 and rotating of the rotating axle 26 and, ultimately, both the oscillation and the rotation functions of the apparatus 10.

The oscillating plate 24 and the rotating axle 26 are preferably driven solely by the motor 38 so that the motor 38 acts as a single drive for both functions of the apparatus 10. It should be noted that the oscillating plate 24 and the rotating axle 26 may be driven via any mechanism apparent to one of ordinary skill in the art. In an exemplary embodiment, the oscillating plate 24 may be driven by an eccentric gear having an eccentric rod thereon that turns and drives a pendulum, which is further connected to the oscillating plate 24. The motor 38 may drive the eccentric gear and cause the eccentric rod to also rotate, which may then be translated into a to-and-fro motion of the pendulum. As the pendulum swivels, the oscillating plate 24 also swivels, causing the oscillating motion to translate to a brush attached to the oscillating plate 24. Likewise, the eccentric gear further works with a secondary gear set to rotate the rotating axle 26, thereby causing the rotating motion to translate to a brush attached to the rotating axle 26. Of course, the present invention may be accomplished via any gear mechanism and should not be limited as described herein. Moreover, the speed of oscillation and the speed of rotation may be carefully controlled by adjusting the speed of the motor 38 and/or the specific gears and other like mechanisms within the apparatus 10.

The present invention may provide oscillating speeds and rotating speeds as needed for effective use as a skin cleanser, whether used as a facial cleanser or a body cleanser. In a preferred embodiment, the present invention may provide a single speed of rotation and oscillation, driven by the motor 38, as described above. Specifically, in a preferred embodiment, the apparatus 10 may provide a cleansing brush with an oscillating speed of between about 14,000 and 20,000 oscillations per minute (“opm”). More preferably, the apparatus 10 may provide a cleansing brush with an oscillating speed of between about 15,000 and 18,000 opm. Moreover, in the preferred embodiment, the apparatus 10 may provide a cleansing brush with a rotating speed of between about 250 and 400 rpm. More preferably, the apparatus 10 may provide a cleansing brush with a rotating speed of about 300 and 380 rpm. Most preferably, the apparatus 10 may provide a cleansing brush with a rotating speed of between about 360 rpm and 370 rpm.

Alternatively, the apparatus 10 of the present invention may have two or more speeds, which may be controlled by the power button 18. For example, pushing the power button 18 once may engage a first speed for the motor 38, pressing the power button 18 a second time may engage the motor at a higher speed, and pressing the power button 18 a third time may shut the apparatus 10 off. For example, the power button may drive the motor at a first speed to output a rotation speed of between about 280 and 320 rpm, and an oscillation speed of about 14,000 opm. Moreover, the power button may drive the motor at a second speed to output a rotation speed of between about 355 and 395 rpm, and an oscillation speed of between about 17,000 and 20,000 opm.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are non-limiting, and it should be noted that claim limitations presented herein are not meant to describe the invention as a whole. Moreover, the invention illustratively disclosed herein suitably may be practiced in the absence of any element which is not specifically disclosed herein.

I claim:

1. A skin cleansing apparatus comprising:
a main body having a power source and a cleansing head on an end thereof, the cleansing head comprising an oscillating plate and a rotating axle for separately driving a cleansing attachment in either an oscillation motion or a rotating motion.

2. The skin cleansing apparatus of claim 1 further comprising:
a battery enclosure within the main body for providing DC power.

3. The skin cleansing apparatus of claim 1 further comprising:
a motor, the motor interconnected to both the oscillating plate and the rotating axle for driving both the oscillating plate and the rotating axle.

4. The skin cleansing apparatus of claim 3 further comprising:
a power switch for engaging the motor.

5. The skin cleansing apparatus of claim 3 further comprising:
a set of mechanical elements to translate rotational motion of the motor into oscillating motion of the oscillating plate.

6. The skin cleansing apparatus of claim 3 further comprising:
a set of mechanical elements to drive rotational motion of the rotating axle.

7. The skin cleansing apparatus of claim 1 further comprising:
a cleansing attachment engaging the oscillating plate.

8. The skin cleansing apparatus of claim 7 wherein the cleansing attachment has an abrasive surface for cleansing a user's skin.

9. The skin cleansing apparatus of claim 1 further comprising:
a cleansing attachment engaging the rotating axle.

10. The skin cleansing apparatus of claim 9 wherein the cleansing attachment has an abrasive surface for cleansing a user's skin.

11. The skin cleansing apparatus of claim 3 wherein the motor drives the oscillation at a first oscillating speed and the rotation at a first rotating speed.

12. The skin cleansing apparatus of claim 11 wherein the motor drives the oscillation at a second oscillating speed and the rotation at a second rotating speed.

13. The skin cleansing apparatus of claim 11 wherein the motor drives the oscillation between about 14,000 and 20,000 oscillations per minute and the rotation between about 250 and 400 rotations per minute.

14. The skin cleansing apparatus of claim 11 wherein the first oscillation speed is about 15,000 oscillations per minute and the first rotation speed is about 360 rpm.

15. The skin cleansing apparatus of claim 12 wherein the first oscillating speed is about 14,000 oscillations per minute and the second oscillating speed is between about 17,000 and 20,000 oscillations per minute, and the first rotating speed is between about 280 and 320 rpm and the second rotating speed is between about 355 and 395 rpm.

16. A method of cleansing skin comprising the steps of:
providing a skin cleansing apparatus comprising a main body having a power source and a cleansing head on an end thereof, the cleansing head comprising an oscillating plate and a rotating axle for separately driving a cleansing attachment in either an oscillation motion or a rotating motion; and
attaching a first cleansing attachment to the oscillating plate for oscillating the first cleansing attachment.

17. The method of claim 16 further comprising the step of:
attaching a second cleansing attachment to the rotating axle for rotating the second cleansing attachment.

18. The method of claim 16 wherein the cleansing attachment comprises a cleansing surface.

19. A method of cleansing skin comprising the steps of:
providing a skin cleansing apparatus comprising a main body having a power source and a cleansing head on an end thereof, the cleansing head comprising an oscillating plate and a rotating axle for separately driving a cleansing attachment in either an oscillation motion or a rotating motion; and
attaching a first cleansing attachment to the rotating axle for rotating the first cleansing attachment.

20. The method of claim 19 further comprising the step of:
attaching a second cleansing attachment to the oscillating plate for oscillating the second cleansing attachment.
